(19) United States
(12) Patent Application Publication
(10) Pub. No.: US 2004/0138641 A1
(43) Pub. Date: Jul. 15, 2004

(54) Contrast dispensing system with nonsticking luer connector

(75) Inventor: Charles R. Patzer, Columbus, OH (US)

Correspondence Address: WOOD, HERRON & EVANS, LLP, 2700 CAREW TOWER, 441 VINE STREET, CINCINNATI, OH, 45202, US

(73) Assignee: Medex, Inc., Dublin, OH

(21) Appl. No.: 10/339,249

(22) Filed: Jan. 9, 2003

(51) Int. Cl. A61B 19/00

(52) U.S. Cl. 604/407

(57) ABSTRACT

A contrast dispensing system (10) including a mating pair of luer connectors (112, 202) to couple a reusable set (100) and a disposable set (200), a mating pair of luer connectors (204, 218) to couple the disposable set (200) to a manifold (16), a pair of luer connectors (230, 232) to couple a control syringe (18) to the manifold (16), and/or a pair of luer connectors (234, 236) to couple the manifold (16) to a patient catheter (238), wherein one or both of the connectors of a pair of coupled connectors (112, 202; 204, 218; 230, 232; 234, 236) is comprised of low surface energy plastic material, such as high density polyethelene, to reduce or eliminate sticking together from expelled (312) contrast media (14).

### BACKGROUND OF THE INVENTION

I. Field of the Invention

The present invention relates to contrast dispensing systems.

II. Description of Prior Art

Contrast dispensing systems are well known and provide a mechanism for selectively coupling a source of contrast media, such as from a pressurized bag or bottle, to a patient through a manifold and control syringe structure. Contrast dispensing systems typically have a spike at one end designed to selectively engage the contrast media source and an outlet luer connector at the other end adapted to attach to one port of the manifold (also in the form of a luer connector shaped to mate with the outlet luer connector). Another luer connector port of the manifold is connected to a control syringe via a luer connector or rotator luer connector associated with the control syringe. The syringe and manifold may be manipulated to pull contrast media into the syringe, and then to expel same back out to the patient through yet another luer connector port which is coupled through a further luer connector to a catheter in the patient.

The media is usually supplied in bulk volume far in excess of that needed for one patient's purposes. To reduce waste, many contrast dispensing systems include a reservoir between the spike and the luer connector to temporarily hold a quantity of the contrast media. A quantity of the contrast media may be loaded into the reservoir from the source. The media is then taken from the reservoir by manipulation of the manifold and syringe to dispense the contrast media to the patient. Thereafter, part or all of the contrast dispensing system may be disposed of and a new system put in place for the next patient so as not to waste the remaining contrast media in the bag or bottle. In an effort to avoid cross-contamination between patients, many such systems include a reusable set carrying the spike coupled to reusable tubing, and a disposable set carrying the outlet luer connector at the end of disposable tubing and a check valve in series with the tubing. These systems also provide a pair of luer connectors on the adjacent ends of the reusable and disposable sets for selectively joining the reusable and disposable sets together. A reservoir may be provided in the disposable set. One large container of contrast media may then be used with multiple patients by switching out the disposable set between patient uses.

When the various luer connectors are joined together, they define generally sealed fluid paths through which the contrast media flows. During change out of parts of the system, either during use with a patient or between patients, one or more of the many pairs of luer connectors are undone. For example, the disposable set may be removed from the reusable set meaning that the reusable luer connector remains to connect to the luer connector of the new disposable set, or the control syringe is removed, leaving the manifold luer connector for attachment to the luer connector of another control syringe. As the remaining and new luer connectors are brought together as a coupled pair, a small amount of the contrast media on the remaining luer connector may be pushed out of the fluid path and come into contact with the plastic of both luer connectors, especially outside of the flow path. The expelled media tends to function as a glue causing the coupled pair of luer connectors to stick together. As a consequence, it is difficult to disengage the pair of luer connectors. The clinician may even have to use hemostats to wrench the two connectors apart.

Many contrast dispensing systems include some length of the disposable tubing extending directly from each of the paired connectors, which allows the clinician some maneuverability with the hemostats to avoid damage to the remaining mating luer connector as it is unstuck from the other luer connector. In some cases, there is little or no tubing, such as with the connectors joining the manifold and control syringe, which makes removal a problem. Similarly, in my application Ser. No. 10/180,730, filed Jun. 26, 2002, and entitled Contrast Dispensing System, I disclose a system that advantageously eliminates the length of tubing between the disposable luer connector which attaches to the reusable set and the reservoir. While there are many advantages to that system, it also makes separation of the stuck mating luer connectors all the more problematic.

### SUMMARY OF THE INVENTION

The present invention provides an improved contrast dispensing system which overcomes the abovementioned stickiness drawbacks of a coupled pair of luer connectors. To this end, and in accordance with the principles of the present invention, one or both of the luer connectors of a given pair of luer connectors is comprised of a plastic that has a low affinity for the contrast media so as to discourage or prevent the expelled media from sticking or knitting the two connectors together. In this regard, it has been conventional to use polycarbonate plastic for the material of the luer connectors. Such plastic has a high affinity for the contrast media, however, and thus actually promotes the sticking together of the paired luer connectors. Luer connectors are available which are comprised of plastic with a lower affinity to the contrast media. Such plastic may be characterized as being of a low surface energy material, and can include polyolefins, nylon and high density polyethelene (HDPE), to name a few. By using a luer connector comprised of such a low surface energy plastic material for either or both of the luer connectors of a coupled pair of luer connectors, they will tend not to become stuck together by the expelled contrast media and so are easier to separate after use.

By virtue of the foregoing, there is thus provided an improved contrast dispensing system which overcomes the tendency of a coupled pair of luer connectors to stick together as occurred with prior contrast dispensing systems. These and other objects and advantages of the present invention shall be made apparent from the accompanying drawings and the description thereof.

### BRIEF DESCRIPTION OF THE DRAWINGS

The accompanying drawings, which are incorporated in and constitutes a part of this specification, illustrate an embodiment of the invention and, together with a general description of the invention given above, and the detailed description of the embodiment given below, serve to explain the principles of the invention.

FIG. 1 is an exploded, schematic view, not to scale, of a contrast dispensing system having a plurality of coupled pairs of luer connectors in accordance with the principles of the present invention; and

FIGS. 2A-2C are cross-sectional views of one of the coupled pairs of luer connectors of FIG. 1 in various stages of assembly for purposes of explaining the principles of the present invention.

### DETAILED DESCRIPTION OF THE DRAWINGS

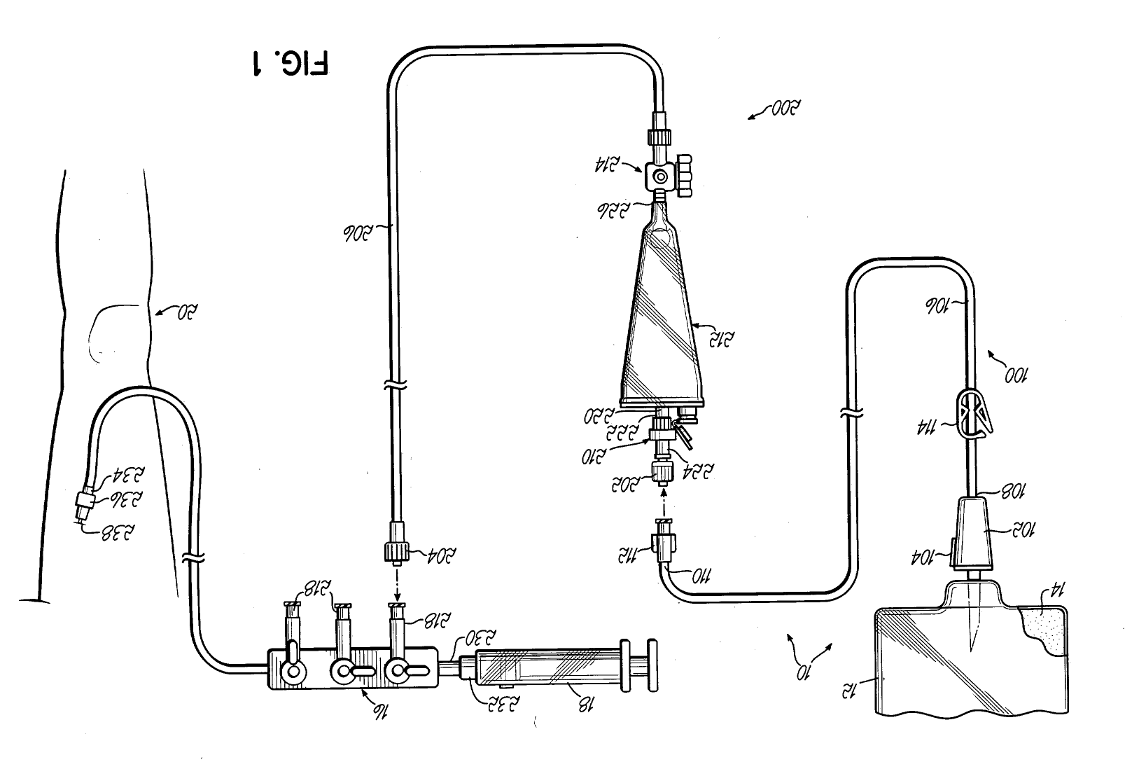

With reference to FIG. 1, there is shown an example of a contrast dispensing system 10 adapted to couple a source 12, such as a bag or bottle, of contrast media 14 to a manifold 16 which, in cooperation with a control syringe 18, administers the media 14 to a patient 20. System 10 is comprised of a reusable set 100 and a disposable set 200 such as generally described in my prior application (Ser. No. 10/180,730, filed Jun. 26, 2002), the disclosure of which has been incorporated herein by reference in its entirety. Sets 100 and 200 may be selectively coupled together at a coupled pair of mating luer connectors 112 and 202 as will be hereinafter described.

Reusable set 100 includes a spike 102 adapted to be selectively coupled in fluid communication with the contrast media 14 to source 12. Spike 102 may include a hinged vent cap 104, as is typical, for venting of spike 102. Cap 104 is shown in the closed state, but may be flipped open for venting. Clear PVC tubing 106 is coupled at one end 108 thereof to spike 102, and at opposite end 110 thereof to a first mating luer connector such as female luer lock 112 for coupling to disposable set 200. Tubing 106 advantageously includes a valve 114 by which to selectively control the flow of fluid through tubing 106 between source 12 and luer connector 112. In the embodiment shown in FIG. 1 the valve 114 is a pinch clamp mounted on tube 106, although other valves may be used such as a stopcock (not shown) inserted in series with the tubing 106. Clamp 114 may either be in an open state as shown in FIG. 1, in which case fluid may flow between spike 102 and connector 112, or may be pinched into the closed state (not shown) in which case tubing 106 is clamped closed to prevent flow of fluid. A vented protector cap (not shown) may also be provided to close off connector 112 when connector 112 is not connected to a second mating luer connector, such as male luer lock 202, of a disposable set 200, for example.

Disposable set 200 is adapted to couple media 14 between connector 202 and the outlet luer connector, such as male luer lock 204, which is adapted to be coupled to one of female luer lock connector ports 218 of manifold 16 so as to facilitate dispensing of contrast media 14 to patient 20. Outlet luer connector 204 and a luer connector port 218 thus also define a coupled pair of luer connectors. Connectors 202 and 204 are coupled together through clear PVC disposable tubing 206. Included in fluid series therewith may be a check valve 210, a reservoir 212, and an optional venting valve 214. Reservoir 212 includes an inlet port 220 to which is directly mounted the outlet side 222 of check valve 210, with the inlet side 224 of valve 210 being directly mounted to mating luer connector 202. Alternatively, a length (not shown) of tubing 206 may extend between either or both of luer connector 202 and check valve 210 on the one hand, and check valve 210 and inlet port 220 of reservoir 212 on the other hand. Reservoir 212 includes a fluid outlet 226 which is coupled to tubing 206 such as through optional venting valve 214. The details of reservoir 212, and its operation, are not otherwise pertinent to the present invention, and reservoir 212 may even be eliminated in some applications.

Manifold 16 includes a syringe port 230 in the form of a female luer lock connector, so that a control syringe 18 may be removably coupled thereto via a male luer lock connector 232, which may be an integral part of syringe 18, a rotator, or at the end of a length of tubing (not shown) between syringe 18 and connector 232. Luer connectors 232 and 230 thus also define a coupled pair of luer connectors. Further, manifold 16 couples to an exhaust luer connector 234 which mates with luer connector 236 of a catheter 238 extending into the limb of a patient 20 such as an arm or thigh. Luer connectors 234 and 236 also define a coupled pair of luer connectors.

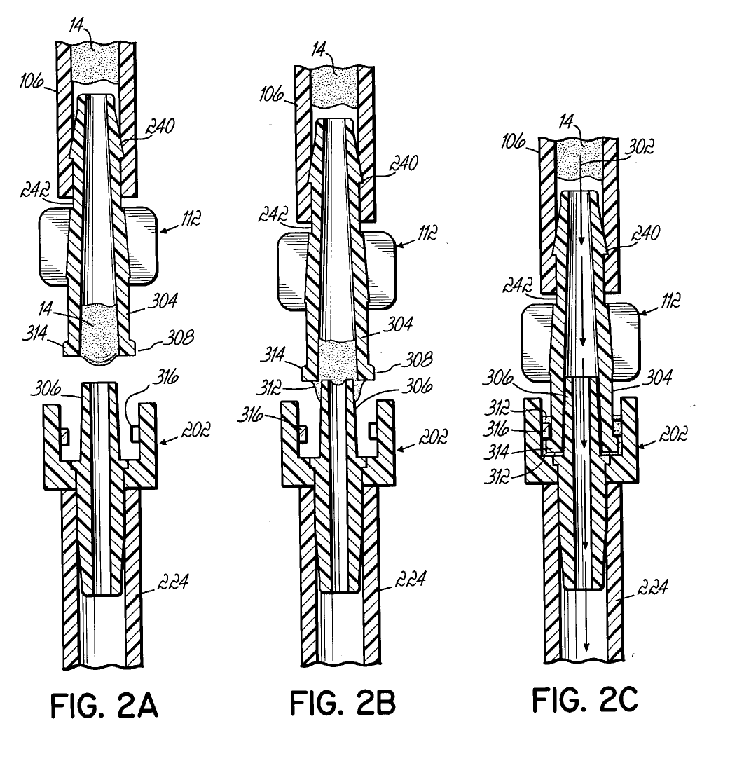

With reference to FIGS. 2A-2C, as can be seen therein, any pair of mating luer connectors may be brought together (connectors 112, 202 are shown, but it will be appreciated that it could be any other pair of mating luer connectors such as pair 204, 218, pair 230, 232, or pair 234, 236) with one of them remaining (e.g., connector 112) and the other being newly provided (e.g., connector 202). When the pair of connectors are coupled together, they define a generally sealed fluid path 302 (FIG. 2C) between the female luer cylinder 304 and male luer taper 306. Before they are coupled together, however, a small amount of media 14 may be hanging off the distal tip end 308 of a remaining connector such as the end of luer cylinder 304 of reusable luer connector 112 (FIG. 2A). As the luer taper 306 of newly-provided connector 202 is brought into contact with the luer cylinder 304 of remaining connector 112, an amount 312 of the media is expelled out of what should be the generally sealed fluid path 302. This expelled media 312 tends to form a glue-like substance which can adhere on the locking flange/threads 314, 316 and/or male and female cylinder segments 304, 306 of the pair of connectors 112 and 202, respectively, especially outside of fluid path 302.

In this regard, it had been conventional practice to use polycarbonate plastic as the material of the luer connectors of system 10, including connectors 112, 202. In practice, polycarbonate plastic can be easily solvent based to tubing 106 and/or 206 as well as the other plastic components of the system such as valve 210, reservoir 212 and/or syringe 18. However, media 14 is of a highly polar nature and has been found to stick to the polycarbonate material, especially outside of the fluid path 302. To overcome this problem, system 10 employs a luer connector(s) comprised of plastic that has a lower affinity for the media 14 for either or both of a pair of mating luer connectors such as one or both of luer connectors 112 and/or 202; 204 and/or 218; 230 and/or 232; or 234 and/or 236. To this end, luer connectors comprised of plastic having a low surface energy material (advantageously less than 37 dynes/cm²) are used. With such plastics, the expelled media 312 does not readily stick to thereto. Hence, if at least one of the connectors of a pair of connectors (such as one of connectors 112, 202) is comprised of the desired low surface energy plastic material, the connectors can be easily unstuck. Thus, if one of connectors 112, 202 is of low surface energy plastic material, the pair of connectors 112, 202, may be readily opened up to disconnect the disposable set 200 from the reusable set 100 without any deleterious or difficult to manage sticking. Similarly, if one of the pairs of connectors 204, 218 is of low surface energy plastic material, manifold 16 and disposable set 200 may be easily disconnected; if one of luer connectors 230 and 232 is of low surface energy plastic material, control syringe 18 may be easily removed from manifold 16 without sticking; and/or if one of connectors 234 and 236 is of low surface energy plastic material, manifold 16 may be easily disconnected from patient catheter 238 without sticking.

Where the connector is made of such low surface energy plastic material, it may not readily bond to the tubing or other plastic components. In that case, the connector may have to be frictionally engaged therewith such as with barbed section 240 on inlet port 242 of the connector (FIG. 2A). If the connector is connector 112, for example, inlet port 242 fits within and grips to the tubing 106 due to the barbed section 240. It is advantageous to make reusable connector 112 of the low surface energy plastic material, and connector 202 as polycarbonate, although that is not required. Advantageously, the low surface energy plastic material is HDPE, although other plastics may also provide the desired reduction in stickiness, including, by way of example, polyolefins, nylon, other density polyethelene, polypropylene, polyalloner, acetene, or FEP (Tetrafluoroethylene Hexafluoropropylene-copolymer).

In use, a reusable set 100 and disposable set 200 are joined at the coupled pair of mating luer connectors 112, 202, disposable set 200 is joined to manifold 16 via outlet luer connector 204 coupled to a luer connector port 218, control syringe 18 is joined to manifold 16 at the coupled pair of luer connectors 230, 232, and the manifold 16 is coupled to the patient via the coupled pair of luer connectors 234, 236. Spike 102 is coupled to a media source 12. The system 10 is primed and reservoir 212 filled, used, or emptied as desired. Valve 214 is manipulated to either couple media 14 from reservoir 212 to manifold 16, or couple tubing 206 to atmosphere for purposes of draining tubing 206, for example. After a patient use, disposable set 200 may be easily removed from reusable set 100 at coupled luer connectors 112 and 202, and a replacement disposable set 200 may be joined to reusable set 100. Also, control syringe 18 may be removed by uncoupling the pair of luer connectors 230 and 232 and a new syringe 18 with its own luer connector 232 coupled to luer connector 230 of manifold 16. Similarly, disposable set 200 may be removed from manifold 16 at the coupled pair of luer connectors 204, 218, and/or manifold 16 removed from the patient at the coupled pair of luer connectors 234, 236.

By virtue of the foregoing, there is thus provided a contrast dispensing system and/or portions thereof which overcomes the sticking problem previously experienced in contrast dispensing systems having disposable and reusable aspects joined at mating luer connectors.

While the present invention has been illustrated by the description of an embodiment thereof, and while the embodiment has been described in considerable detail, it is not intended to restrict or in any way limit the scope of the appended claims to such detail. Additional advantages and modifications will readily appear to those skilled in the art. For example, one connector of the pair of connectors could be made of one type of low surface energy plastic material while the other connector iof the pair is made of another type of such plastic material. Further, one of the connectors could be of polycarbonate as conventional, while the other is of the desired low surface energy plastic material. When a luer connector port 218 and/or syringe luer port 230 are of the low surface energy plastic material, the entire manifold 16 may be formed of that material. Similarly, if desired, the syringe 18 and its luer connector 232 may be formed of the low surface energy plastic material. The invention in its broader aspects is therefore not limited to the specific details, representative apparatus and methods and illustrative examples shown and described. Accordingly, departures may be made from such details without departing from the scope or spirit of applicant's general inventive concept.

Having described the invention, what is claimed is:

1. A disposable set for a contrast dispensing system comprising:
a mating luer connector adapted to be releasably coupled to a luer connector of a reusable set;
an outlet luer connector adapted to be coupled to a manifold system for dispensing contrast to a patient;
disposable tubing coupled to the outlet luer connector;
a reservoir coupled between the mating luer connector and the tubing; and
the mating luer connector being comprised of low surface energy plastic material.

2. The disposable set of claim 1, the mating luer connector being comprised of high density polyethelene.

3. The disposable set of claim 1, the mating luer connector being a male luer lock.

4. The disposable set of claim 1 further comprising a check valve.

5. The disposable set of claim 4, the check valve directly coupled to the mating luer connector and the reservoir having an inlet port directly coupled to the check valve.

6. A reusable set for a contrast dispensing system comprising:
a spike;
reusable tubing coupled at one end to the spike;
a reusable luer connector coupled to another end of the reusable tubing; and
the reusable luer connector being comprised of low surface energy plastic material.

7. The reusable set of claim 6, the reusable luer connector being comprised of high density polyethylene.

8. The reusable set of claim 6, the reusable luer connector being a female luer lock.

9. The reusable set of claim 6 further comprising a valve associated with the reusable tubing.

10. A contrast dispensing system comprising:
a reusable set having a spike adapted to couple to a source of contrast, a first mating luer connector and reusable tubing coupling the spike to the first mating luer connector;
a disposable set having a second mating luer connector adapted to be removably coupled to the first mating luer connector to selectively join the disposable set to the reusable set, the disposable set further including an outlet luer connector adapted to be coupled to a manifold system for dispensing contrast to a patient, and disposable tubing extending between the second mating luer connector and the outlet luer connector; and
at least one of the first and second mating luer connectors being comprised of low surface energy plastic material.

11. The contrast dispensing system of claim 10, the low surface energy plastic material being high density polyethelene.

12. The contrast dispensing system of claim 10, the first mating luer connector being comprised of low surface energy plastic material.

13. The contrast dispensing system of claim 12, the second mating luer connector being comprised of low surface energy plastic material.

14. The contrast dispensing system of claim 10, the second mating luer connector being comprised of low surface energy plastic material.

15. The contrast dispensing system of claim 10, the first and second mating luer connectors being a female luer lock and a male luer lock, respectively.

16. The contrast dispensing system of claim 10, the disposable set further having a check valve between the second mating luer connector and the outlet luer connector.

17. The contrast dispensing system of claim 10, the disposable set further having a reservoir between the second mating luer connector and the outlet luer connector.

18. The contrast dispensing system of claim 17, the disposable set further having a check valve directly coupled to the second mating luer connector and the reservoir having an inlet port directly coupled to the check valve.

19. A contrast dispensing system comprising:
a reusable set having a spike adapted to couple to a source of contrast, a first mating luer connector and reusable tubing coupling the spike to the first mating luer connector;
a disposable set having a second mating luer connector adapted to be removably coupled to the first mating luer connector to selectively join the disposable set to the reusable set, the disposable set further including an outlet luer connector adapted to be coupled to a manifold system for dispensing contrast to a patient, and disposable tubing extending between the second mating luer connector and the outlet luer connector;
a manifold including a plurality of luer connector ports at least one of which is adapted to couple to the outlet luer connector of the disposable set, and a syringe luer connector port adapted to couple to the luer connector of a control syringe;
a control syringe having a luer connector; and
wherein the first and second mating luer connectors can be coupled together as a pair of coupled luer connectors, the outlet luer connector and one of the luer connector ports can be coupled as a pair of coupled luer connectors, and the syringe luer connector port and the luer connector of the syringe can be coupled together as a pair of coupled luer connectors, at least one of the connectors of at least one pair of the coupled luer connectors being comprised of low surface energy plastic material.

20. The contrast dispensing system of claim 19, the low surface energy plastic material being high density polyethelene.

21. The contrast dispensing system of claim 19, the at least one pair being the first and second mating luer connectors, the first mating luer connector being comprised of a low surface energy plastic material.

22. The contrast dispensing system of claim 20, the second mating luer connector being comprised of low surface energy plastic material.

23. The contrast dispensing system of claim 19, the at least one pair being the first and second mating luer connectors, the second mating luer connector being comprised of low surface energy plastic material.

24. The contrast dispensing system of claim 19, the first and second mating luer connectors being a female luer lock and a male luer lock, respectively.

25. The contrast dispensing system of claim 19, the at least one pair being one of the luer connector ports and the outlet luer connector, the luer connector port being comprised of low surface energy plastic material.

26. The contrast dispensing system of claim 25, the outlet luer connector being comprised of low surface energy plastic material.

27. The contrast dispensing system of claim 19, the at least one pair being one of the luer connector ports and the outlet luer connector, the outlet luer connector being comprised of low surface energy plastic material.

28. The contrast dispensing system of claim 19, the at least one pair being the syringe luer connector port and the luer connector of the control syringe, the syringe luer connector port being comprised of low surface energy plastic material.

29. The contrast dispensing system of claim 28, the luer connector of the syringe being comprised of low surface energy plastic material.

30. The contrast dispensing system of claim 19, the at least one pair being the syringe luer connector port and the luer connector of the control syringe, the luer connector of the syringe being comprised of low surface energy plastic material.

31. The contrast dispensing system of claim 19, at least one connector of each pair of the coupled luer connectors being comprised of low surface energy plastic material.

32. The contrast dispensing system of claim 19, the manifold being comprised of low surface energy plastic material.

33. The contrast dispensing system of claim 19, the control syringe and its luer connector being integrally formed of low surface energy plastic material.

34. The contrast dispensing system of claim 19 further comprising a manifold exhaust luer connector adapted to couple to a catheter luer connector to define a further pair of coupled luer connectors.

35. The contrast dispensing system of claim 19, the disposable set further having a check valve between the second mating luer connector and the outlet luer connector.

36. The contrast dispensing system of claim 19, the disposable set further having a reservoir between the second mating luer connector and the outlet luer connector.

37. The contrast dispensing system of claim 36, the disposable set further having a check valve directly coupled to the second mating luer connector and the reservoir having an inlet port directly coupled to the check valve.
